(19) United States
(12) Patent Application Publication
(10) Pub. No.: US 2021/0361688 A1
(43) Pub. Date: Nov. 25, 2021

(54) SYSTEM, METHOD AND USE OF A CERTAIN MEDICATION FOR REDUCING VIRAL REPLICATION IN THE AIRWAYS MUCOSAE

(71) Applicant: Carlos Alberto RIVEROS, Coral Gabels, FL (US)

(72) Inventor: Carlos Alberto RIVEROS, Coral Gabels, FL (US)

(21) Appl. No.: 17/327,306

(22) Filed: May 21, 2021

Related U.S. Application Data
(60) Provisional application No. 63/182,125, filed on Apr. 30, 2021.
(60) Provisional application No. 63/028,714, filed on May 22, 2020.

(51) Int. Cl. A61K 31/7048; A61K 31/573; A61K 9/00; A61P 31/14

(52) U.S. Cl. CPC A61K 31/7048; A61P 31/14; A61K 9/0078; A61K 31/573

(57) ABSTRACT

A system, method, use, combination and kits useful in the administration of a certain medication for reducing viral replication of certain viruses during the early stage of transmission or as a prophylaxis when high risk of exposure to a virus is detected or predicted, administering efficiently a high local concentration of the certain medication while minimizing systemic exposure. Specifically it refers to a system, a method, a use, pharmaceutical combination and pharmaceutical kits of a certain nebulized medication to reduce viral replication. The development uses inhalers or nebulizers to deliver at least one certain medication directly into the upper and lower airways mucosae.

### CROSS-REFERENCE TO RELATED APPLICATIONS

This application claims the benefit of U.S. Provisional Application No. 63/028,714, filed 22 May 2020, and U.S. Provisional Application No. 63/182,125, filed 30 Apr. 2021 which are expressly incorporated herein by reference in its entirety.

### TECHNICAL FIELD

The disclosure contained herein generally relates to systems, methods, uses, combinations and kits useful for the treatment of diseases caused by viral replication in the upper and lower airways mucosae such as COVID-19.

### BACKGROUND

In about November to December 2019 a novel coronavirus was identified as the cause of pneumonia cases in Wuhan (China). It spread, resulting in an epidemic throughout China, and thereafter in other countries throughout the world. In February 2020, the World Health Organization designated the disease COVID-19, which stands for coronavirus disease 2019. The virus is also known as severe acute respiratory syndrome coronavirus 2 (SARS-CoV-2) (1).

COVID-19 is a betacoronavirus in the same subgenus as the severe acute respiratory syndrome (SARS) virus (as well as several bat coronaviruses), but in a different clade. The structure of the receptor-binding gene region is very similar to that of the SARS coronavirus, and the virus has been shown to use the same receptor, the angiotensin converting enzyme 2 (ACE2), for cell entry (2).

In the situation of rapidly increasing cases, inappropriate management of mild cases could increase the burden of healthcare system and medical costs. Viral clearance is a major standard in the assessment of recovery and discharge from medical care, but early results illustrated that the persistence of viral RNA is heterogeneous despite the rapid remission of symptoms and can last over three weeks even in very mild cases. In addition, long hospitalization stays may increase the risk for hospital-associated mental health problems and unexpected hospital-acquired infections. (9)

At the beginning, the outbreak identified an initial association with a seafood market that sold live animals in Wuhan, China. However, as the outbreak progressed, person-to-person spread became the main mode of transmission.

Person to person transmission is thought to occur mainly via respiratory droplets, resembling the spread of influenza. With droplet transmission, the virus is released in respiratory secretions when an infected person breathes, coughs, sneezes, or talks, and can infect another person if such secretions make direct contact with the mucous membranes. Infection can also occur if a person touches an infected surface and then touches his or her eyes, nose, or mouth. Droplets typically do not travel more than six feet (about two meters) and do not linger in the air. There is still controversy about this topic.

Whether SARS-CoV-2 can be transmitted through the airborne route (through particles smaller than droplets that remain in the air over time and distance) under natural conditions has been controversial.

Reflecting the current uncertainty regarding transmission mechanisms, recommendations on airborne precautions in the health care setting vary by location; airborne precautions are universally recommended when aerosol-generating procedures are performed.

It appears that SARS-CoV-2 can be transmitted prior to the development of symptoms and throughout the course of illness. However, most data informing this issue is from studies evaluating viral RNA detection from respiratory and other specimens, and detection of viral RNA does not necessarily indicate the presence of infectious virus.

A study suggested infectiousness started 2.3 days prior to symptom onset, peaked 0.7 days before symptom onset, and declined within seven days; however, most patients were isolated following symptom onset, which would reduce the risk of transmission later in illness regardless of infectiousness. These findings raise the possibility that patients might be more infectious in the earlier stage of infection, but additional data is needed to confirm this hypothesis (3).

How long a person remains infectious is also uncertain. The duration of viral shedding is variable; there appears to be a wide range, which may depend on severity of the illness. In one study of 21 patients with mild illness (no hypoxia), 90 percent had repeated negative viral RNA tests on nasopharyngeal swabs by 10 days after the onset of symptoms; tests were positive for longer in patients with more severe illness (4). In contrast, in another study of 56 patients with mild to moderate illness (none required intensive care), the median duration of viral RNA shedding from nasal or oropharyngeal specimens was 24 days, and the longest was 42 days (5). However, as mentioned above, detectable viral RNA does not always correlate with isolation of infectious virus, and there may be a threshold of viral RNA level below which infectivity is unlikely. In the study of nine patients with mild COVID-19 described above, infectious virus was not detected from respiratory specimens when the viral RNA level was <106 copies/mL (6).

Risk of transmission from an individual with SARS-CoV-2 infection varies by the type and duration of exposure, use of preventive measures, and likely individual factors (e.g., the amount of virus in respiratory secretions).

Antibodies against the virus are induced in those who have become infected. Preliminary evidence suggests that some of these antibodies are protective, but this remains to be definitively established. It is unknown whether all infected patients develop a protective immune response and how long any protective effect will last.

Diagnosis of COVID-19 is made by detection of SARS-CoV-2 RNA by reverse transcription polymerase chain reaction (RT-PCR). Various RT-PCR assays are used around the world; different assays amplify and detect different regions of the SARSCoV-2 genome. Common gene targets include nucleocapsid (N), envelope (E), spike (S), and RNA-dependent RNA polymerase (RdRp), as well as regions in the first open reading frame (7).

Serologic tests detect antibodies to SARS-CoV-2 in the blood, and those that have been adequately validated can help identify patients who have had COVID-19. However, sensitivity and specificity are still not well defined. Detectable antibodies generally take several days to weeks to develop, for example, up to 12 days for IgM and 14 days for IgG (8).

### SUMMARY OF THE INVENTION

The present invention provides a method, system, use, combinations and kits useful in the administration of certain medications for reducing viral replication of certain viruses in the upper and lower airways mucosae during early stage of the disease or as prophylactic when high risk of exposure is detected or predicted. Depending on the medication, later stages of the disease can also be addressed.

### BRIEF DESCRIPTION OF THE DRAWINGS

FIG. 1 shows the results of the average subgenomic RNA load for two groups of patients: patients that received the treatment of Example 2 (named TREATMENT) and patients who received the best standard of care treatment (named BSC). The X axis corresponds to the days in which the samples were collected (days 0, 3, 5 and 7), and the Y axis corresponds to subgenomic RNA load (copies/mL).

### DETAILED DESCRIPTION

A system for administering, a method for reducing viral replication, the use of a nebulized medication in the treatment of certain viruses in the airway mucosae, as well as combinations and kits useful in said treatment were developed and are described herein. The system, method, use and associated combinations and kits including the certain medication are useful during an early stage of the disease for reducing viral replication or as prophylactic when high risk of exposure to the virus is detected or predicted. Specifically, the system, method, use and associated combinations and kits comprise administering a certain medication to reduce viral replication. The development uses inhalers or nebulizers to administer the certain medication into the upper and lower airways mucosae.

The system for administering a therapeutically effective dose of a certain medication for reducing viral replication in the upper and lower airways mucosae comprise administering said certain medication in a device for delivering a therapeutically effective dose of said certain medication directly into the lungs in the form of an inhalable mist or inhalable form. An inhalable mist is a suspension of a finely divided liquid in a gas which can be inhaled by a subject in need.

As mentioned above, it is also described a method for reducing viral replication in a subject in need thereof comprising administering an inhalable mist of a therapeutically effective dose of a certain medication in into the upper and lower airways mucosae.

The system for administering, method for reducing viral replication and use of a certain medication mentioned above reduce viral replication caused by respiratory virus. The term respiratory virus is understood in this application as a virus in which viral replication occurs in the respiratory track. Therefore, viruses that are transmitted similar to COVID-19 and have some degree of response to the certain medication are considered as respiratory virus, for example, RNA viruses, MERS, MERS-CoV, SARS-CoV, SARS-CoV-1, and influenza, wherein the RNA viruses use importin (IMP) α/β1 and are selected from DENV 1-4, West Nile Virus, Venezuelan equine encephalitis virus (VEEV) and influenza.

The combinations and kits mentioned above include the certain medications useful in reducing viral replication caused by a respiratory virus, as well as additional anti-inflammatory drugs.

Considering that in the initial transmission of the virus, the virus infects the surface of the upper airways, followed by subsequent spread to the lower airways, the inventor has discovered that nebulization, inhalation or intranasal administration are suitable routes of administration for a solution of certain medication such as ivermectin, wherein the amount of ivermectin available in the upper and lower airways may be enough to reduce the initial replication of the virus in the airways. This action would, as a consequence, reduce the viral replication in early phases of the infection and this will also represent a lower viral load. Thus, for an individual, the delivery of ivermectin should minimize the severity of the disease.

Given that these administration routes have shown promising results with nebulized ivermectin, it is expected that viral replication would reduce (in comparison with the first taken sample in the subject) with other molecules, since it would likewise facilitate that the molecules are directly delivered at the target site and their mechanism of action in an adequate amount.

The certain medication is selected from the group consisting of ivermectin, nitazoxinide, chloroquine, hydroxychloroquine, selamectin, doramectin, eprinomectin, abamectin, remdesivir, nafamostat, molnupiravir, ampligen, amantadine, umifenovir, umifenovir, moroxydine, oseltamivir, peramivir, rimantadine, baloxavir marboxil, zanamivir, bamlanivimab, bamlanivimab/etesevimab combination therapy, lopinavir, ritonavir, lopinavir-ritonavir combination therapy, casirivimab, imdevimab, tocilizumab, etesevimab, VIR-7831, EXO-CD24, PF-07321332, MIR-19, and siRNAs molecules, or combinations thereof. The siRNA molecules include siLuc, siN-2, siN-3, siN-4, siR-7, siR-8, siR-9, siR-10, siR11, siR-12, siR-13, siR-14, and siR-15.

The term “transmission” as used herein is commonly defined as any skilled artisan will know, and includes the transmission to other subject. It is also considered that the method, system and use prevent viral replication within the same subject, i.e., prophylaxis. Viral replication is thought to be mainly through upper airways and mucosa, including alveolus, lung or bronchi.

Although in vitro data provides robust evidence of the different medications against virus, some of their known routes of administration (e.g., oral, intramuscular), do not include a correlation with clinically achievable plasma and lung concentration thereof. However, nebulization, inhalation or intranasal administration thereof could achieve enough concentration in the surface of the upper airways during early phase of transmission.

The inventor has found that the use of a certain medication already known and that has been tested in vitro to reduce viral replication could be administered by a different route (e.g., nebulized or inhaled) to reduce viral replication in the upper and lower airways mucosae during the early stage of the disease or as a prophylaxis when high risk of exposure is detected or predicted. More importantly, the nebulized, inhaled or intranasal presentation could have lower possibility of side effects by reducing the amount of medication in serum/blood, and at the same time providing a good level of contact of the medication with the virus during the period of early installation and replication phase in upper and lower airways.

The system for administering a therapeutically effective dose and the method for reducing viral replication described below can deliver the certain medication directly into the lungs, wherein the certain medication is further combined with other drugs, such as anti-inflammatory agents.

The anti-inflammatory agent is selected from, but is not limited to, baricitinib, dexamethasone, prednisone, prednisolone methylprednisolone, betamethasone, and beclametasone.

Additional components such as surfactants, propellants, solvents, cosolvents, cryoprotectants and/or buffer salts are pharmaceutically acceptable excipients included in the solution including the certain medication in order to achieve proper nebulization. The pharmaceutically acceptable excipients included but are not limited to: glycerol, propylene glycol, glycerin, and polyethylene glycol.

Also disclosed is the use of a nebulized certain medication in the treatment of a disease caused by viral replication. It is also disclosed the use of a nebulized certain medication for the preparation of a medicament useful in the treatment of a disease caused by viral replication.

In said uses, the certain medication is as defined above, but can also be combined with anti-inflammatory agents. The anti-inflammatory agent is selected from, but is not limited to, baricitinib, dexamethasone, prednisone, prednisolone methylprednisolone, betamethasone, and beclametasone. The nebulized certain medication alone or in combination with other molecules or medications is also useful in preventing transmission of the virus.

The method of using inhalers or nebulizers, preferably with disposable components, for administering a therapeutically effective dose of a medication having good in vitro activity against the SARS-CoV-2 (COVID19) virus is applicable or adaptable to these and other medications as well.

By virtue of the system and method described herein, and the use of disposable components it is possible to administer the certain medication to large numbers of people. As a result, a significant reduction of severe cases needing ventilatory support and less fatal cases can be expected.

The system, method and uses described herein could be used for preventing development of a disease after contact or risk of contact including but not limited to health workers, elderly people, persons exposed to public, airplanes among others.

Ivermectin and Antivirals

Ivermectin is a globally used medication approved by the Food and Drug Administration (FDA) for treating of parasite infections. This drug has been used in humans and animals. In the near past, it was investigated its use to treat viruses during previous epidemic events (12).

Originally identified as an inhibitor of the interaction between the human immunodeficiency virus-1 (HIV-1) integrase protein (IN) and the importin (IMP) α/β1 heterodimer responsible for IN nuclear import (13), ivermectin has since been confirmed to inhibit IN nuclear import and HIV-1 replication (14). Other uses of ivermectin have been reported (15), but ivermectin has been shown to inhibit nuclear import of host and viral proteins (16), including simian virus SV40 large tumor antigen (T-ag) and dengue virus (DENV) non-structural protein 5 (13,14). More importantly, it has been demonstrated to limit infection by RNA viruses such as DENV 1-4 (17), West Nile Virus (18), Venezuelan equine encephalitis virus (VEEV) (19) and influenza (20), this broad-spectrum activity is believed to be due to the reliance by many different RNA viruses on IMPα/β1 during infection (21)(22). Ivermectin has similarly been shown to be effective against the DNA virus pseudorabies virus (PRV) both in vitro and in vivo, with ivermectin treatment shown to increase survival in PRV-infected mice (23).

Recently Caly et al. reported in vitro activity of ivermectin against SARS-CoV-2 following a single addition to Vero-hSLAM cells, and suggest that these data “demonstrate that ivermectin is worthy of further consideration as a possible SARS-CoV-2 antiviral” (25) In isolation, these in vitro data is robust and encouraging but, as mentioned above, this report does not include a correlation of the in vitro findings with clinically achievable plasma concentrations and, more relevantly, lung concentrations, that would permit the determination of whether the macrocyclic lactones (and specifically in this case, ivermectin) are genuine therapeutic options.

Caly et al. bathed Vero-hSLAM cells with ivermectin at a concentration of 5 μM from 2 hours post-infection with SARS-CoV-2 isolate Australia/VIC01/2020 until the conclusion of the experiment. SARS-CoV-2 RNA was determined by RT-PCR at Days 0 to 3 in both supernatant and cell pellet experiments. The authors noted 93 to 99.8% reduction in viral RNA for ivermectin versus DMSO control at 24 h in supernatant (released virions) and cell associated viral RNA (total virus) respectively. They also describe a 5000 fold reduction of viral RNA by hour 48 and maintenance of that effect at 72 hours. Additional experiments were conducted with serial dilutions of ivermectin to establish the concentration-response profile, and the authors describe ivermectin as a potent inhibitor of SARS-CoV-2, with an IC₅₀ determined to be about 2 μM under these conditions (26).

While the findings by Caly et al. are promising, there is no evidence that the 5 μM concentration of ivermectin used by Caly et al. in the in vitro SARS-CoV-2 experiment, can be achieved in vivo. The pharmacokinetics of ivermectin in humans are well described, and even with the highest reported dose of approximately 1700 μg/kg (i.e., 8.5 times the FDA approved dose of 200 μg/kg), the maximum plasma concentration was only 0.28 μM. This is 18 times less than the concentration required to reduce SARS-CoV-2 viral replication in vitro. The accumulation of ivermectin in the tissues is slight and would not be sufficient to achieve the antiviral effect with conventional doses. Although high doses of ivermectin in adults or children are well tolerated, the clinical effects of ivermectin at a concentration of 5 μM are unknown and may be associated with toxicity. Consequently, ivermectin has an in vitro activity against SARS-CoV-2, but this effect is unlikely to be observed in vivo at known doses.

However, as demonstrated in the Examples below, in the system, method and used disclosed above, when the certain medication is ivermectin, it is administered 3 times a day for 5 days. Furthermore, when the certain medication is ivermectin, it is administered at a dose of 3 to 6 mL, or 3 to 5 mL, every 8 hours for 5 days. When the certain medication is ivermectin, the liquid solution is in a concentration between 0.1 and 3%, preferably 1%.

Finally, when the certain medication is ivermectin, and it is administered in combination with an antiviral such as dexamethasone, they are administered at a proportion of 10:1, respectively.

Pharmaceutical Kit and Pharmaceutical Combination

The term “pharmaceutical kit” or “pharmaceutical combination” as used herein, means the pharmaceutical composition or compositions that are used to administer the certain medication(s), and/or the certain medication(s) combined with anti-inflammatory agents. When the certain medication(s) and anti-inflammatory agents are administered simultaneously, the pharmaceutical kit or pharmaceutical combination can contain the certain medication(s) and anti-inflammatory agents in a single pharmaceutical composition, or in separate pharmaceutical compositions. When the compounds are not administered simultaneously, the pharmaceutical kit or combination will contain the certain medication(s) and anti-inflammatory agents in separate pharmaceutical compositions. The pharmaceutical kit or combination comprises the certain medication(s) and anti-inflammatory agents in separate pharmaceutical compositions in a single package or in separate pharmaceutical compositions in separate packages.

In one embodiment, the pharmaceutical kit or combination comprises the components: a certain medication in association with a pharmaceutically acceptable carrier; and another certain medication in association with a pharmaceutically acceptable carrier. In another embodiment, the pharmaceutical kit or combination comprises the following components: a certain medication in association with a pharmaceutically acceptable carrier; and another certain medication in association with a pharmaceutically acceptable carrier wherein the components are provided in a form which is suitable for sequential, separate and/or simultaneous administration.

In one embodiment, the pharmaceutical kit or combination comprises the components: a certain medication and an anti-inflammatory agent in a single pharmaceutical composition in association with a pharmaceutically acceptable carrier. In another embodiment, the pharmaceutical kit or combination comprises the components: a certain medication in association with a pharmaceutically acceptable carrier; and an anti-inflammatory agent in association with a pharmaceutically acceptable carrier. In yet another embodiment, the pharmaceutical kit or combination comprises the components: a certain medication in association with a pharmaceutically acceptable carrier; and an anti-inflammatory agent in association with a pharmaceutically acceptable carrier, wherein the components are provided in a form which is suitable for sequential, separate and/or simultaneous administration.

In yet another embodiment, the pharmaceutical kit or combination comprises: a first container comprising a certain medication, in association with a pharmaceutically acceptable carrier; and a second container comprising another certain medication in association with a pharmaceutically acceptable carrier, and a container means for containing said first and second containers. In yet another embodiment, the pharmaceutical kit or combination comprises: a first container comprising a certain medication, in association with a pharmaceutically acceptable carrier; and a second container comprising an anti-inflammatory agent, in association with a pharmaceutically acceptable carrier, and a container means for containing said first and second containers.

The pharmaceutical kit or combination also includes at least one container with a fixed dose of the given drug to be administered via nebulization. In one embodiment, the pharmaceutical kit or combination comprises a plurality of containers with the determined drug or the combination of at least one specific drug, at least one anti-inflammatory agent and at least one pharmaceutically acceptable vehicle, for example 3 containers (ampoules or vials type) of 10 mL that allows the administration of 3 doses of the drug determined every 8 hours to the patient.

The “pharmaceutical kit” or “pharmaceutical combination” can also be provided by instructions, such as dosage and administration instructions. Such dosage and administration instructions can be of the kind that is provided to a doctor, for example by a drug product label, or they can be of the kind that is provided by a doctor, such as instructions to a patient.

A pharmaceutical combination of a therapeutically effective dose of a certain medication for reducing viral replication in the upper and lower airways mucosae selected from the group consisting of ivermectin, nitazoxinide, chloroquine, hydroxychloroquine, selamectin, doramectin, eprinomectin, abamectin, remdesivir, nafamostat, molnupiravir, ampligen, amantadine, umifenovir, umifenovir, moroxydine, oseltamivir, peramivir, rimantadine, baloxavir marboxil, zanamivir bamlanivimab, lopinavir, ritonavir, casirivimab, imdevimab, tocilizumab, etesevimab, VIR-7831, EXO-CD24, PF-07321332, MIR-19, and siRNAs molecules or combinations thereof; and, an anti-inflammatory drug selected from the group consisting of baricitnib, dexamethasone, prednisone, and methylprednisolone or combinations thereof. In an embodiment, the certain medication in the pharmaceutical combination is ivermectin, and the anti-inflammatory drug is dexamethasone.

A pharmaceutical kit allowing for the simultaneous, sequential or separate administration of: a certain medication for reducing viral replication in the upper and lower airways mucosae selected from the group consisting of ivermectin, nitazoxinide, chloroquine, hydroxychloroquine, selamectin, doramectin, eprinomectin, abamectin, remdesivir, nafamostat, molnupiravir, ampligen, amantadine, umifenovir, umifenovir, moroxydine, oseltamivir, peramivir, rimantadine, baloxavir marboxil, zanamivir bamlanivimab, lopinavir, ritonavir, casirivimab, imdevimab, tocilizumab, etesevimab, VIR-7831, EXO-CD24, PF-07321332, MIR-19, and siRNAs molecules or combinations thereof; and, an anti-inflammatory drug selected from the group consisting of baricitnib, dexamethasone, prednisone, and methylprednisolone or combinations thereof.

Devices Used to Deliver Medication into the Lungs

Inhalers and nebulizers are the two most common devices used to deliver medication directly into the lungs. In public settings, the devices used to deliver medication may include disposable components to allow a delivery device to be quickly re-used to deliver medication to another person.

Nebulization of a certain medication solution is a common method of generating aerosols. To deliver a certain medication by nebulization, is possible that the certain medication must first be dispersed in a liquid medium (usually aqueous). After the application of a dispersing force (either a gas jet or ultrasonic waves), the certain medication particles are contained within the aerosol droplets, which are then inhaled. The formulation of the certain medication solution is generally designed to optimize the solubility and stability of the certain medication.

A nebulizer is a drug delivery device that can dispense medication directly into the lungs in the inhalable form or as an inhalable mist. The nebulizer machine uses a mixture of processes involving oxygen, compressed air, and even ultrasonic power to atomize and vaporize the liquid medication or solution into small aerosol droplets, or a mist, that can be inhaled directly into the lungs, alveoli or bronchi.

Nebulizers convert liquid medications into aerosols (mist or inhalable form), which are a suspension of liquid particles in gas. In the nebulizers the certain medication appears as mist that is inhaled by the patient in need thereof and delivered directly to the lungs. The size of the droplet or particle depends on the construction of the nebulizer and the air pressure, but generally varies between 0.5 and 10 μm or between 2 and 5 μm.

There are two types of nebulizers available for consumers, tabletop or portable nebulizers. Tabletop nebulizers are heavy, and they are not meant to be carried around and need an electric outlet for operation. Portable nebulizers on the other hand can be carried around easily and are light weight devices.

Portable nebulizers are handheld devices that are designed to deliver the certain medication when patients are both outdoors and inside a home or public place. A portable nebulizer typically includes: a system to convert the liquid certain medication into mist; a nebulizer cup or receptacle to hold the medication; and a mouthpiece or a mask to inhale the certain medication. In the system of the present invention, the mouthpiece and or mask may include disposable components. The drugs placed in the receptacle are inhaled by the patient in the form of a mist which directly reaches the lungs.

### EXAMPLES

### Example 1. Ivermectin 1%

Subjects infected with SARS CoV 2 that qualified to be included in the trial, received ivermectin 1% administered via nebulization during the early phase of the infection. The ivermectin was administered in a dose of 3 mL (0.03 g) every 8 hours, during 5 days at home isolated but supervised actively via telemedicine.

This system for administering reduced the viral replication as measured by subgenomic mRNA and consequently the load of the active SARS-CoV-2 virus in the upper and lower respiratory tract by more than 90%, resulting in significant clinical improvement including the severity of the disease and duration.

### Example 2. Ivermectin 1% and Dexamethasone Administration

An ivermectin solution for nebulization was prepared by mixing 3 mL of ivermectin 1% (10 mg/mL, provided by Vecol, Bogota Colombia https://vecol.com.co/) with 0.3 mL (1.2 mg) of dexamethasone solution (at 4 mg/mL), formal glycerol and propylene glycol.

3 mL of the solution was administered to the subject directly into the lungs in the form of an inhalable mist. Given that approximately only 10% of the nebulized administered solution will reach the respiratory pathways, each nebulization distributed approximately 3 mg of ivermectin into an average of 150 cc of dead space and probably some alveolar space, delivering approximately 0.02 mg per cc, which was above the IC₅₀ concentration necessary to inhibit viral replication (IC₅₀=0.00175 mg/cc). During Phase 1, it was demonstrated that these doses did not cause changes in Pulmonary Function Tests in healthy individuals.

The ivermectin combined with dexamethasone was administered by a nebulizer 3× day during 5 days.

A statistically significant decrease in viral replication measured by subgenomic mRNA was observed after administration of the ivermectin and dexamethasone combination in comparison with a placebo.

### Example 3. Preliminary Data

14 outpatients in early stages of SARS-CoV-2 disease (considering “early stage” of the disease to the first day that the patient realizes that he/she is positive for the virus or a within the first three days after starting symptoms) who expressed at least one of the following genes: Gen E, Gen N and Gen RdRp under the Charité Foundation protocol, were subjected to the treatment described in Example 2. Under the same study, the viral replication of 7 different outpatients treated with the best supportive care (BSC) treatment was also evaluated. Among the different BSC treatments, patients were treated with acetaminophen, anti-inflammatory agents, bronchodilator agents, among others. More details of the protocol used can be found in trial No. NCT04595136 registered at https://clinicaltrials.gov/.

To evaluate if the treatment of Example 2 was useful for reducing the virus' replication capacity compared to the BSC treatment in all the evaluated outpatients, a brushing sample of the nasopharyngeal zone was taken on days 0, 3, 5, and 7, and the genetic material (in this case RNA) was extracted from said samples.

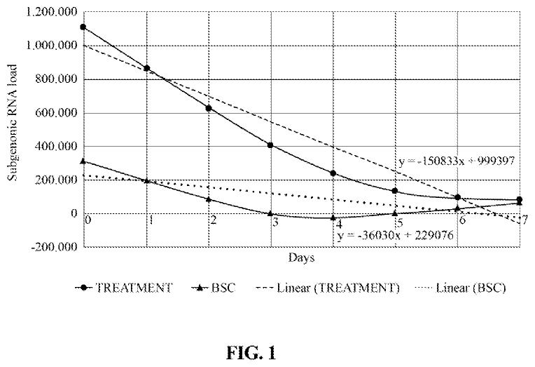

RNA was extracted from the samples using the VN143 Viral RNA Mini Kit (Genolution). The method was modified from published methods for detecting coronavirus subgenomic mRNA. The purified RNA was reverse transcribed using SuperScript II (ThermoFisher Scientific, https://www.thermofisher.com) and a SARS-CoV-2 specific primer (WHSA-29950R: 5′-TCTCCTAAGAAGCTATTAAAAT-3′). The complementary DNA obtained was subjected to qPCR (40 cycles at 94° C. for 30 s, 56° C. for 30 s and 72° C. for 1.5 min. Optimized condition to amplify small subgenomic mRNA) and AmpliTaq Gold DNA Polymerase (ThermoFisher Scientific) with primers (FAM WHSA-00025F: 5′-CCAACCAACTTTCGATCTCTTGTA-3′BHQ1 and FAM WHSA-29925R: 5′-ATGGGGATAGCACTACTAAAATTA-3′BHQ1) (Perera et al., 2020 Emerging Infectious Diseases). Quantification was carried out with a plasmid where the amplicon fragment was inserted in 4 known concentrations (100, 1,000, 10,000 and 1,000,000 copies/ml). Results obtained are illustrated in FIG. 1 (averages of all patients).

From the results obtained, researchers noted that within the group of BSC patients, some showed an increase in their symptoms, others a decrease (i.e., improvement), some were static or their condition had worsened at the end of the treatment. These results confirmed that there was not a single standard behavior or a general pattern within this group.

Regarding the group of patients treated according to Example 2 (TREATMENT in FIG. 1), a clear tendency was observed demonstrating that in all the evaluated cases there was a reduction in RNA load, i.e., the virus' replication capacity was reduced over time. These positive results allowed researches to conclude that if the treatment of Example 2 is carried out at early stages of the SARS-CoV-2 disease, the replication capacity of the virus is diminished.

When comparing the average viral replication load of subgenomic RNA achieved in each group (FIG. 1), it can be concluded that the TREATMENT group steeper slope when compared to the results of the BSC group, demonstrating a faster reduction in the replication capacity of the virus in patients treated according to Example 2 when compared to BSC treatment.

The following references are incorporated into this description by reference:
1. World Health Organization. Director-General's remarks at the media briefing on 2019-nCoV on 11 Feb. 2020. https://www.who.int/dg/speeches/detail/who-directorgeneral-s-remarks-at-the-media-briefing-on-2019-ncov-on-11-february-2020 (Accessed on Feb. 12, 2020).
2. Zhou P, Yang X L, Wang X G, et al. A pneumonia outbreak associated with a new coronavirus of probable bat origin. Nature 2020; 579:270.
3. He X, Lau E H Y, Wu P, et al. Temporal dynamics in viral shedding and transmissibility of COVID-19. Nat Med 2020.
4. Liu Y, Yan L M, Wan L, et al. Viral dynamics in mild and severe cases of COVID-19. Lancet Infect Dis 2020
5. Xiao A T, Tong Y X, Zhang S. Profile of RT-PCR for SARS-CoV-2: a preliminary study from 56 COVID-19 patients. Clin Infect Dis 2020.
6. Wölfel R, Corman V M, Guggemos W, et al. Virological assessment of hospitalized patients with COVID-2019. Nature 2020.
7. World Health Organization. Laboratory testing for 2019 novel coronavirus (2019-nCoV) in suspected human cases. https://www.who.int/publications-detail/laboratory-testingfor-2019-novel-coronavirus-in-suspected-human-cases-20200117 (Accessed on Apr. 22, 2020).
8. Zhao J, Yuan Q, Wang H, et al. Antibody responses to SARS-CoV-2 in patients of novel coronavirus disease 2019. Clin Infect Dis 2020.
9. Than H M et al., Management of mild cases of COVID-19 in low-resource countries: An experience in Vietnam, Journal of Microbiology, Immunology and Infection, https://doi.org/10.1016/j.jmii.2020.04.012
10. Wang Y, Zhang D, Du G, et al. Remdesivir in adults with severe COVID-19: a randomised, double-blind, placebo-controlled, multicentre trial. Lancet 2020.
11. Mitjà O, Clotet B. Use of antiviral drugs to reduce COVID-19 transmission. Lancet Glob Health 2020; 8:e639.
12. Gotz et al., 2016; Lundberg et al., 2013; Tay et al., 2013; Wagstaff et al., 2012)
13. Wagstaff, K. M., et al., 2011. An AlphaScreen®-based assay for high-throughput screening for specific inhibitors of nuclear import. J. Biomol. Screen 16 (2), 192-200.
14. Wagstaff, K. M., et al., 2012. Ivermectin is a specific inhibitor of importin alpha/betamediated nuclear import able to inhibit replication of HIV-1 and dengue virus. Biochem. J. 443 (3), 851-856.
15. Mastrangelo, E., et al., 2012 August Ivermectin is a potent inhibitor of flavivirus replication specifically targeting NS3 helicase activity: new prospects for an old drug. J. Antimicrob. Chemother. 67 (8), 1884-1894.
16. Kosyna, F. K., et al., 2015. The importin alpha/beta-specific inhibitor Ivermectin affects HIF-dependent hypoxia response pathways. Biol. Chem. 396 (12), 1357-1367.
17. Tay, M. Y., et al., 2013. Nuclear localization of dengue virus (DENV) 1-4 non-structural protein 5; protection against all 4 DENV serotypes by the inhibitor Ivermectin. Antivir. Res. 99 (3), 301-306.
18. Yang, S. N. Y., et al., 2020. The broad spectrum antiviral ivermectin targets the host nuclear transport importin alpha/beta1 heterodimer. Antivir. Res. 104760.
19. Lundberg, L., et al., 2013. Nuclear import and export inhibitors alter capsid protein distribution in mammalian cells and reduce Venezuelan Equine Encephalitis Virus replication. Antivir. Res. 100 (3), 662-672.
20. Gotz, V., et al., 2016. Influenza A viruses escape from M×A restriction at the expense of efficient nuclear vRNP import. Sci. Rep. 6, 23138.
21. Caly, L., Wagstaff, K. M., Jans, D. A., 2012. Nuclear trafficking of proteins from RNA viruses: potential target for anti-virals? Antivir. Res. 95, 202-206.
22. Jans, D. A., Martin, A. J., Wagstaff, K. M., 2019. Inhibitors of nuclear transport. Curr. Opin. Cell Biol. 58, 50-60.
23. Lv, C., et al., 2018. Ivermectin inhibits DNA polymerase UL42 of pseudorabies virus entrance into the nucleus and proliferation of the virus in vitro and vivo. Antivir. Res. 159, 55-62.
24. Hiscox, J. A., et al., 2001. The coronavirus infectious bronchitis virus nucleoprotein localizes to the nucleolus. J. Virol. 75 (1), 506-512.
25. Caly, L., Wagstaff, K. M., Jans, D. A., 2012. Nuclear trafficking of proteins from RNA viruses: potential target for anti-virals? Antivir. Res. 95, 202-206.
26. Caly et all Antiviral Research 178 (2020) 104787.

1. A system for administering a therapeutically effective dose of a certain medication for reducing viral replication in the upper and lower airways mucosae comprising administering the certain medication in a device for delivering a therapeutically effective dose of said certain medication in the form of an inhalable mist.

2. The system of claim 1, wherein the certain medication is selected from ivermectin, nitazoxinide, chloroquine, hydroxychloroquine, selamectin, doramectin, eprinomectin, abamectin, remdesivir, nafamostat, molnupiravir, ampligen, amantadine, umifenovir, umifenovir, moroxydine, oseltamivir, peramivir, rimantadine, baloxavir marboxil, zanamivir bamlanivimab, lopinavir, ritonavir, casirivimab, imdevimab, tocilizumab, etesevimab, VIR-7831, EXO-CD24, PF-07321332, MIR-19, and siRNAs molecules or combinations thereof.

3. The system of claim 1, wherein the certain medication is further combined with anti-inflammatory drugs selected from baricitnib, dexamethasone, prednisone, and methylprednisolone.

4. The system of claim 2, wherein the certain medication is ivermectin.

5. The system of claim 2, wherein the ivermectin is administered 3 times a day for 5 days, at a dose of 3 mL every 8 hours for 5 days and wherein the ivermectin is in a concentration between 0.1 and 3%.

6-7. (canceled)

8. The system of claim 1, wherein the viral replication is caused by a virus selected from RNA viruses, MERS, MERS-CoV, SARS-CoV, SARS-CoV-1, SARS-CoV-2 and influenza.

9-10. (canceled)

11. A method for reducing viral replication in a subject in need thereof comprising administering an inhalable mist of a therapeutically effective dose of a certain medication into the upper and lower airways mucosae.

12. The method of claim 11, wherein the certain medication is selected from ivermectin, nitazoxinide, chloroquine, hydroxychloroquine, selamectin, doramectin, eprinomectin, abamectin, remdesivir, nafamostat, molnupiravir, bamlanivimab, lopinavir, ritonavir, casirivimab, imdevimab, tocilizumab, etesevimab, VIR-7831, EXO-CD24, PF-07321332, MIR-19, and siRNAs molecules or combinations thereof.

13. The method of claim 11, wherein the certain medication is administered by an aerosol inhaler.

14. The method of claim 12, wherein the certain medication is ivermectin.

15. (canceled)

16. The method of claim 11, wherein the inhalable mist has a particle size between 0.5 and 10 μm.

17. The method of claim 11, wherein the certain medication is further combined with an anti-inflammatory drug selected from baricitnib, dexamethasone, prednisone, and methylprednisolone.

18. The method of claim 11, wherein the viral replication is caused by a virus selected from RNA viruses, MERS, MERS-CoV, SARS-CoV, SARS-CoV-1, SARS-CoV-2 and influenza.

19. The method of claim 11, wherein the certain medication is administered during the early stage of the disease.

20. The method of claim 18, wherein the RNA viruses use importin (IMP) α/β1 and are selected from DENV 1-4, West Nile Virus, Venezuelan equine encephalitis virus (VEEV) and influenza.

21. The method of claim 14, wherein the ivermectin is administered 3 times a day for 5 days, at a dose of 3 mL every 8 hours for 5 days and wherein the ivermectin is in a concentration between 0.1 and 3%.

22-25. (canceled)

26. Use of a nebulized certain medication for the preparation of a medicament useful in the treatment of a disease caused by viral replication in the upper and lower airways mucosae, wherein the certain medication is selected from ivermectin, nitazoxinide, chloroquine, hydroxychloroquine, selamectin, doramectin, eprinomectin, abamectin, remdesivir, nafamostat, molnupiravir, bamlanivimab, lopinavir, ritonavir, casirivimab, imdevimab, tocilizumab, etesevimab, VIR-7831, EXO-CD24, PF-07321332, MIR-19, and siRNAs molecules or combinations thereof.

27.-28. (canceled)

29. A pharmaceutical combination of a therapeutically effective dose of:
a certain medication for reducing viral replication in the upper and lower airways mucosae selected from the group consisting of ivermectin, nitazoxinide, chloroquine, hydroxychloroquine, selamectin, doramectin, eprinomectin, abamectin, remdesivir, nafamostat, molnupiravir, ampligen, amantadine, umifenovir, umifenovir, moroxydine, oseltamivir, peramivir, rimantadine, baloxavir marboxil, zanamivir bamlanivimab, lopinavir, ritonavir, casirivimab, imdevimab, tocilizumab, etesevimab, VIR-7831, EXO-CD24, PF-07321332, MIR-19, and siRNAs molecules or combinations thereof; and,
an anti-inflammatory drug selected from the group consisting of baricitnib, dexamethasone, prednisone, and methylprednisolone or combinations thereof.

30. The pharmaceutical combination of claim 29, where the certain medication is ivermectin and the anti-inflammatory drug is dexamethasone.

31. A pharmaceutical kit allowing for the simultaneous, sequential or separate administration of:
a certain medication for reducing viral replication in the upper and lower airways mucosae selected from the group consisting of ivermectin, nitazoxinide, chloroquine, hydroxychloroquine, selamectin, doramectin, eprinomectin, abamectin, remdesivir, nafamostat, molnupiravir, ampligen, amantadine, umifenovir, umifenovir, moroxydine, oseltamivir, peramivir, rimantadine, baloxavir marboxil, zanamivir bamlanivimab, lopinavir, ritonavir, casirivimab, imdevimab, tocilizumab, etesevimab, VIR-7831, EXO-CD24, PF-07321332, MIR-19, and siRNAs molecules or combinations thereof; and,
an anti-inflammatory drug selected from the group consisting of baricitnib, dexamethasone, prednisone, and methylprednisolone or combinations thereof.
